(19) United States
(12) Patent Application Publication
(10) Pub. No.: US 2024/0351981 A1
(43) Pub. Date: Oct. 24, 2024

(54) NOVEL BISMALEIMIDE COMPOUNDS HAVING IMPROVED SOLUBILITY AND THEIR USE IN CURABLE COMPOSITIONS

(71) Applicant: Evonik Operations GmbH, Essen (DE)

(72) Inventors: Dr. Sergey EVSYUKOV, Ludwigshafen (DE); Dr. Sabine LEICK, Ronneburg (DE); Dr. Stephan Kohlstruk, Gladbeck (DE)

(73) Assignee: Evonik Operations GmbH, Essen (DE)

(21) Appl. No.: 18/294,243

(22) PCT Filed: Jul. 22, 2022
(86) PCT No.: PCT/EP2022/070573

§ 371 Date: Feb. 1, 2024

(30) Foreign Application Priority Data
Aug. 2, 2021 (EP) 21189164.3

(51) Int. Cl. C07D 207/448; C08F 222/40

(52) U.S. Cl. CPC C07D 207/448; C08F 222/402; C08F 2810/20

(57) ABSTRACT

Specific bismaleimide compounds are made, and curable compositions are made that include at least one of these bismaleimides and at least one specific polyimide. A process is developed for the manufacture of these curable compositions, and crosslinked polymers are obtainable by this process. A process is developed for the manufacture of a composite material by curing a mixture of a fibrous or particulate reinforcement and the curable composition or the crosslinked polymer as well as the obtained composite material.

The present invention relates to specific bismaleimide compounds, curable compositions comprising at least one of these bismaleimides and at least one specific polyimide. Furthermore, the present invention refers to a process for the manufacture of these curable compositions, and crosslinked polymers obtainable by this process. Finally, the present invention pertains to a process for the manufacture of a composite material comprising curing a mixture of a fibrous or particulate reinforcement and the curable composition or the crosslinked polymer of the present invention as well as the obtained composite material.

Commercially available and known bismaleimide (BMI) monomers including aliphatic ones are known to possess poor solubility. Therefore, in order to make solvent-based formulations with high resin content being used in the production of printed circuit boards, the use of prepolymerized or chain-extended BMIs was necessary, which implies additional production costs, toxic chain-extending agents, and increased viscosity of the solution (Evsyukov, et al., Curr. Trends Polym. Sci, 2020, 20, 1-28). Alternatively, highly toxic and high-boiling amide solvents can be used to a certain extent (the solubility of BMIs is limited even in these powerful solvents).

For the economic production of the solution-processed prepregs and fiber-reinforced laminates therefrom, highly soluble aliphatic BMIs are required. Up to now, no alternative aliphatic BMIs are known that would possess high solubility and provide high heat resistance. The dimer BMIs, also known as X-BMI, based on branched aliphatic C₃₆-dimer diamines (DeFusco, et al., NWC Tech. Publ. 6543, Naval Weapons Center, China Lake, California, USA, 1984; Dershem, et al, U.S. Pat. No. 7,102,015, 2006), are well soluble in organic solvents, but their heat resistance in cured form is low because of a large distance between functional maleimide groups. Thus, cured resins based on X-BMIs were reported to show Tg (glass transition temperature) in the range of 60-95° C., which is approx. 200° C. lower than that of classical BMI resins (Gouzman, et al., Adv. Mater. Technol., 2019, 4, 1900368; Evsyukov, et al., Curr. Trends Polym. Sci, 2020, 20, 1-28).

Moreover, for the production of BMIs and BMI/co-monomer products, it is desirable to gain improved processability of solution-based BMI resins. Due to increasing restriction of applications of toxic solvents of the amide type, which are typical BMI process solvents according to the state of the art, there is a need for the development of resins processable from conventional low-boiling, preferably below 120° C., more preferably below 100° C., solvents.

Furthermore, improved solubility also could also provide better compatibility with other monomers and co-monomers in hot melt formulations.

Therefore, it was an objective of the present invention to provide BMIs with high solubility, preferably at least 30%, more preferably at least 34%, in, preferably at least three, low-boiling solvents.

It has been surprisingly found that the use of a specific propane-1,3-diamines as a starting material in the classical BMI synthesis (reaction with maleic anhydride followed by cyclodehydration) results in the formation of highly soluble bismaleimides of formula (I) that can for example be used in solution-based BMI formulations in conventional low-boiling solvents without pre-polymerization or chain-extension, as well as in hot melt formulations. High solubility in conventional, low-boiling solvents allow highly concentrated solutions without pre-polymerization or chain-extension.

In the invention 2-(3,3,5-trimethylcyclohexyl)propane-1,3-diamine was used as an exemplarily compound for the specific propane-1,3-diamines. 2-(3,3,5-trimethylcyclohexyl)propane-1,3-diamine has been described to be suitable for use as hardener in epoxy resin compositions by (i) reaction of isophorone with malononitrile and (ii) Cobalt alloy-catalyzed hydrogenation of 2-(3,5,5-trimethylcyclohex-2-en-1-ylidene) malononitrile (II). Partial hydrogenation of II by reaction of II in THF with H₂ in the presence of Pd/alumina at 75° and 50 bar H₂ for 5 h and completion of hydrogenation od the resulting product solution using a cobalt alloy containing cobalt: 75.9% by wt., aluminum: 20.0% by wt., chromium: 1.5% by wt. and nickel 2.6% by wt. at 100° and 100 bar H₂ for 5 h, providing 2-(3,3,5-trimethylcyclohexyl)propane-1,3-diamine in 76% yield (EP 3255035 A1).

Moreover, 2-(3,3,5-Trimethylcyclohexyl)propane-1,3-diamine is used as a curing agent in an epoxy resin composition comprising (a) epoxy resin, (b) crosslinking agent consisting of 0.1-100 wt. % 2-(3,3,5-trimethylcyclohexyl)propane-1,3-diamine and 0-99.9 wt. % other diamines or/and polyamines, (c) 0.1-10 wt. % other crosslinking catalysts, (d) optionally ≥1 crosslinking precursor and (e) optionally other additives (EP 3255079 A1).

### Definitions

As used herein, including in the accompanying claims, the terms, which are collectively used, have the following meanings, if not explicitly stated otherwise.

As used herein, the term “curable” means that an original compound(s) or mixture material(s) can be transformed into a solid, substantially non-flowing material for example by means of chemical reaction, crosslinking, or radiation crosslinking.

As used herein, the term “mixture” means a physical or mechanical aggregation or a combination of two or more individual, chemically distinct compounds that are not chemically united.

As used herein, the term “co-monomer” means a compound that can undergo polymerization or copolymerization, thereby contributing constitutional units to the essential structure of a polymer.

As used herein, the term “co-monomer component” means one co-monomer or a mixture of two or more co-monomers, preferably one co-monomer or a mixture of two to four co-monomers.

As used herein, the term “alkenylphenol” means organic compounds comprising at least one alkenyl-substituted phenol group. The term “alkenylphenol” comprises alkenylphenols, wherein two phenol groups are bridged via a difunctional group, e.g. alkenylbisphenols. Examples include 2,2′-diallyl-bisphenol A.

As used herein, the term “alkenylphenyl ether” means organic compounds comprising at least one alkenyloxyphenyl group, i.e. an ether group wherein the ether oxygen atom is connected on one hand to an alkenyl residue and on the other hand to a phenyl residue. The term “alkenylphenyl ether” comprises alkenylphenyl ethers, wherein two phenyl groups are bridged by a difunctional group, e.g. alkenylbisphenol ether. Examples include diallyl ether of bisphenol A.

As used herein, the term “alkenylphenol ether” means organic compounds comprising at least one alkenylphenoxy group, e.g. an ether group wherein the ether oxygen atom is connected on one hand to an alkenylphenyl group and on the other hand to a an alkyl or an aryl group. The term “alkenylphenol ether” comprises organic compounds, wherein two alkenylphenoxy groups are bridged by a difunctional group, e.g. by an aromatic group such as a benzophenone group. Examples include bis-(o-propenylphenoxy) benzophenone.

As used herein, the term “polyamine” means an organic compound having two or more primary amino groups —NH_{2.} Examples include, but are not limited to 4,4′-diaminodiphenylmethane, 4,4′-diaminodiphenylsulfone, 3,3′-diaminodiphenylsulfone, diaminodiphenylindane, m-phenylenediamine, p-phenylenediamine, 2,4-diaminotoluene, 2,6-diaminotoluene, m-xylylenediamine and aliphatic diamines such as ethylenediamine, hexamethylenediamine, trimethylhexamethylenediamine, 1,12-diaminododecane.

As used herein, the term “aminophenol” means amino-substituted phenols. Examples include m-aminophenol and p-aminophenol.

As used herein, the term “amino acid hydrazides” means any hydrazides of amino acids. Examples include m-aminobenzhydrazide and p-aminobenzhydrazide.

As used herein, the term “cyanate ester” means a bisphenol or polyphenol, e.g. novolac, derivative, in which the hydrogen atom of the phenolic OH group is substituted by a cyano-group, resulting in an —OCN group. Examples include bisphenol A dicyanate ester, commercially available as, e.g. Primaset BADCy from Lonza or AroCy B-10 from Huntsman, as well as other Primaset or AroCy types, e.g. bis(3,5-dimethyl-4-cyanatophenyl)methane (AroCy M-10), 1,1-bis(4-cyanatophenyl)ethane (AroCy L-10), 2,2-bis(4-cyanatophenyl)-1,1,1,3,3,3-hexafluoropropane (AroCy F-10), 1,3-bis(1-(4-cyanatophenyl)-1-methylethylidene)benzene (AroCy XU-366), di (4-cyanatophenyl)thioether (AroCy RDX-80371; AroCy T-10), bis(4-cyanatophenyl)dichloromethylidenemethane (AroCy RD98-228), bis(4-cyanatophenyl)octahydro-4,7-methanoindene (AroCy XU-71787.02L), as well as bis(4-cyanatophenyl)methane, bis(3-methyl-4-cyanatophenyl)methane, bis(3-ethyl-4-cyanatophenyl)methane, di(4-cyanatophenyl)ether, 4,4-dicyanatobiphenyl, 1,4-bis(1-(4-cyanatophenyl)-1-methylethylidene)benzene, resorcinol dicyanate. A preferred example is bisphenol A dicyanate ester.

Any bond intersected by a bracket indicates a bond that connects the moiety within the bracket to other moieties of the same compound. For example, in the group shown below the two bonds of the ethenyl group intersected by the bracket on the right side connect this moiety to other moieties of the compound containing this ethenyl group

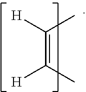

As used herein, the term “halogen” means a fluorine, chlorine, bromine or iodine atom, preferably a fluorine or chlorine atom, more preferably a fluorine atom.

As used herein, “alkyl” means a straight-chain or branched alkyl group. The term “alkyl with n to m carbon atoms” means an alkyl group with n to m carbon atoms. If not denoted otherwise, “alkyl” means an alkyl with 1 to 6 carbon atoms. In the context of the present invention, preferred alkyl groups are straight-chain or branched alkyl groups with up to 4 carbon atoms. Examples of straight-chain and branched alkyl groups include, but are not limited to methyl, ethyl, propyl, isopropyl, butyl, isobutyl, tert.-butyl, the isomeric pentyls, the isomeric hexyls, preferably methyl and ethyl and most preferred methyl.

As used herein, “alkylene” means a difunctional alkyl group. The term “alkylene with n to m carbon atoms” means an alkylene group with n to m carbon atoms. If not denoted otherwise, “alkylene” means an alkylene with 1 to 12 carbon atoms. In the context of the present invention, preferred alkylene groups are alkylene groups with 1 to 9 carbon atoms, more preferably from 1 to 6 carbon atoms. Examples include, but are not limited to methylene, ethylene, propylene, butylene, hexamethylene and 2,2,4-trimethylhexamethylene. Particularly preferred is 2,2,4-trimethylhexamethylene.

As used herein, “alkenylene” means a difunctional alkenyl group. The term “alkenylene with n to m carbon atoms” means an alkenylene group with n to m carbon atoms. If not denoted otherwise, “alkenylene” means an alkenylene with 2 to 12 carbon atoms. In the context of the present invention, preferred alkenylene groups are alkenylene groups with 2 to 10 carbon atoms, more preferably from 2 to 6 carbon atoms. Examples include, but are not limited to ethenylene, propenylene, and butenylene. Particularly preferred is ethenylene.

As used herein, “alkoxy” means a straight-chain or branched alkyl group, which is bonded to the compound via an oxygen atom (—O—). The term “alkoxy with n to m carbon atoms” means an alkoxy with n to m carbon atoms. If not denoted otherwise, “alkoxy” means a straight-chain or branched alkyl group with up to 6 carbon atoms. In the context of the present invention, preferred alkoxy groups are straight-chain or branched alkoxy groups with up to 4 carbon atoms.

As used herein, “alkenyl” means a straight-chain or branched hydrocarbon group comprising a carbon-carbon double bond. The term “alkenyl with n to m carbon atoms” means an alkenyl with n to m carbon atoms. If not denoted otherwise, “alkenyl” means a straight-chain or branched hydrocarbon group comprising a carbon-carbon double bond in any desired position and 2 to 10 carbon atoms. In the context of the present invention, preferred alkenyl groups comprise a carbon-carbon double bond in any desired position and 2 to 6, more preferably 2 to 4 carbon atoms. Examples of alkenyl groups include, but are not limited to ethenyl, 1-propenyl, 2-propenyl, isopropenyl, 1-butenyl, 2-butenyl, 3-butenyl and isobutenyl. Preferred examples are 1-propenyl and 2-propenyl.

As used herein the term “monocarbocyclic group” means a “monocarbocyclic aliphatic group” or a “monocarbocyclic aromatic group”.

As used herein the term “dicarbocyclic group” means a “dicarbocyclic aliphatic group” or a “dicarbocyclic aromatic group” group.

As used herein the term “monocarbocyclic aliphatic group” means a cycloalkylene group.

As used herein, “cycloalkyl” means a monofunctional carbocyclic saturated ring system. The term “cycloalkyl with n to m carbon atoms” means a cycloalkyl with n to m carbon atoms. Preferably cycloalkyl means a cycloalkyl group with 5 to 6 carbon atoms. Examples of cycloalkyl groups include, but are not limited to cyclopropanyl, cyclobutanyl, cyclopentanyl, cyclohexanyl, cycloheptanyl or cyclooctanyl, preferably cyclopentanyl and cyclohexanyl.

As used herein, “cycloalkylene” means a difunctional carbocyclic saturated ring system. The term “cycloalkylene with n to m carbon atoms” means a cycloalkylene with n to m carbon atoms. If not denoted otherwise, “cycloalkylene” means a cycloalkylene group with 3 to 8 carbon atoms. In the context of the present invention preferred cycloalkylene groups are cycloalkylene groups with 5 to 7, more preferably 5 or 6 carbon atoms. Examples include, but are not limited to cyclopropylene, cyclobutylene, cyclopentylene, cyclohexylene, cycloheptylene or cyclooctylene, preferably cyclopentylene and cyclohexylene.

As used herein, “dicarbocyclic aliphatic group” means a difunctional bicyclic condensed, bridged or fused saturated ring system. If not denoted otherwise, “dicarbocyclic aliphatic group” means a difunctional bicyclic condensed, bridged or fused saturated ring system with 9 to 20 carbon atoms. Examples include, but are not limited to decalinyl, hydrindanyl and norbornyl.

As used herein, the term “mono-or dicarbocyclic aromatic group” means a difunctional mono-or dicyclic aromatic system, preferably with 6 to 12 carbon atoms, preferably a monocyclic aromatic system. Examples include, but are not limited to, toluene, phenylene, naphthylene, tetrahydronaphthylene, indenylene, indanylene, pentalenylene, fluorenylene and the like, preferably toluene, phenylene or indanylene.

As used herein, the term “aryl” means a monofunctional mono-or dicyclic aromatic system, preferably with 6 to 12 carbon atoms, preferably a monocyclic aromatic system. Examples include, but are not limited to, toluyl, phenyl, naphthyl, tetrahydronaphthyl, indenyl, indanyl, pentalenyl, fluorenyl and the like, preferably toluyl, phenyl or indanyl.

As used herein, the term “heterocyclic group” means a “heterocyclic aliphatic group” or a “heterocyclic aromatic group”.

As used herein, the term “heterocyclic aliphatic group” means a difunctional saturated ring system which, in addition to carbon atoms, comprises one, two or three atoms selected from nitrogen, oxygen and/or sulfur. Preferred heterocyclic aliphatic groups are those containing 3 to 5 carbon atoms and one nitrogen, oxygen or sulfur atom.

As used herein, the term “heterocyclic aromatic group” means a monocyclic aromatic 5-or 6-membered ring, which comprises one, two or three atoms selected from nitrogen, oxygen and/or sulfur, or a bicyclic aromatic group comprising two 5-or 6-membered rings, in which one or both rings can contain one, two or three atoms selected from nitrogen, oxygen or sulfur. Examples include, but are not limited to pyridyl, pyrazinyl, pyrimidinyl, pyridazinyl, oxazolyl, oxydiazolyl, isoxazolyl, thiadiazolyl, tetrazolyl, pyrazolyl, imidazolyl, thiazolyl, thienyl, quinolinyl, isoquinolinyl, cinnolinyl, pyrazolo[1,5-a]pyridyl, imidazo[1,2-a]pyridyl, quinoxalinyl, benzothiazolyl, benzotriazolyl, indolyl, indazolyl.

As used herein the term “bridged multicyclic group” means a group consisting of at least two groups selected from monocarbocyclic aromatic groups, dicarbocyclic aromatic groups, cycloalkylene groups; wherein these groups are linked to each other by direct carbon-carbon bonds or by divalent groups.

Preferred divalent groups are oxy-group, thio-group, alkylene-group with 1 to 3 carbon atoms, sulfone-group, methanone-group, and the following groups:

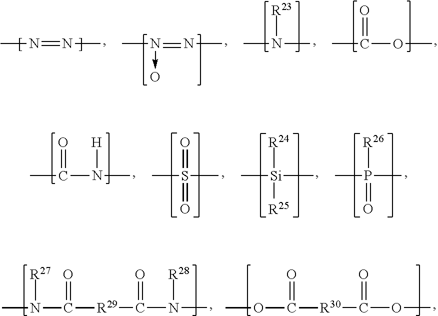

wherein R²³ to R²⁸ are independently selected from alkyl groups with 1 to 6 carbon atoms; and R²⁹ and R³⁰ are independently selected from alkylene groups with 1 to 6 carbon atoms.

In one embodiment the term “bridged multicyclic group” means a group consisting of two monocarbocyclic aliphatic groups, which are linked to each other by a direct carbon-carbon bond or by a divalent group such as oxy-group, thio-group, alkylene-group with 1 to 3 carbon atoms, sulfone-group, methanone-group, or one of the following groups:

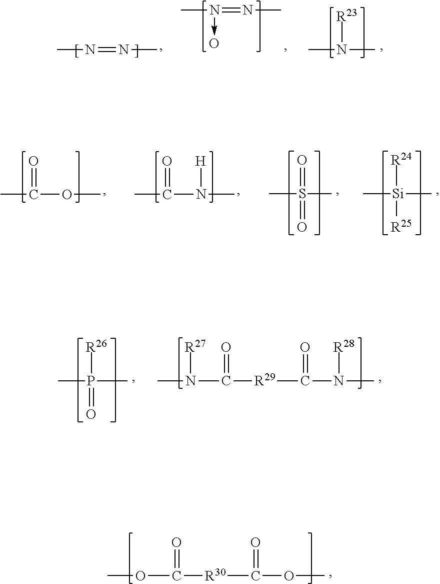

wherein R²³ to R²⁸ are independently selected from alkyl groups with 1 to 6 carbon atoms; and R²⁹ and R³⁰ are independently selected from alkylene groups with 1 to 6 carbon atoms.

In one embodiment the term “bridged multicyclic group” means a group consisting of two cyclohexylene groups, which are linked to each other by a direct carbon-carbon bond or by a divalent group such as oxy-group, thio-group, alkylene-group with 1 to 3 carbon atoms, sulfone-group, methanone-group.

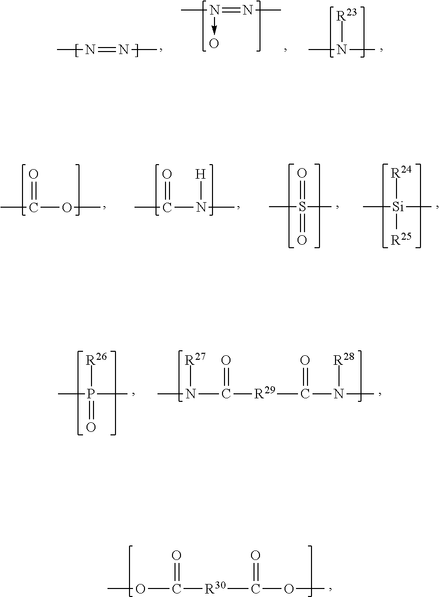

wherein R²³ to R²⁸ are independently selected from alkyl groups with 1 to 6 carbon atoms; and R²⁹ and R³⁰ are independently selected from alkylene groups with 1 to 6 carbon atoms

In one embodiment the term “bridged multicyclic group” means a group consisting of two phenylene groups, which are linked to each other by a direct carbon-carbon bond or by a divalent group such as oxy-group, thio-group, alkylene-group with 1 to 3 carbon atoms, sulfone-group, methanone-group.

As used herein, the addition of the terms “unsubstituted” or “substituted” means that the respective groups are unsubstituted or carry from 1 to 4 substituents selected from alkyl, alkoxy and halogen. Preferred substituents are methyl or ethyl.

As used herein, the terms “x-functional group”, “y-functional group”, “y′-functional group” and “y″-functional group” respectively, denote a group, which is bonded to the remainder of the compound via x, y, y′, or y″ bond(s), respectively. Preferably, the “x-functional group”, “y-functional group”, “y′-functional group” and “y″-functional group” is a difunctional group, i.e. x, y, y′ and y″ are preferably 2.

As used herein, the term “difunctional group” means a group, which is bonded to the remainder of the compounds via two bonds. Difunctional groups include but are not limited to, difunctional aliphatic groups and difunctional aromatic groups. Difunctional aliphatic groups include but are not limited to the following groups:

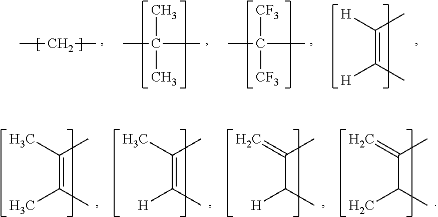

Difunctional aromatic groups include but are not limited to the following groups:

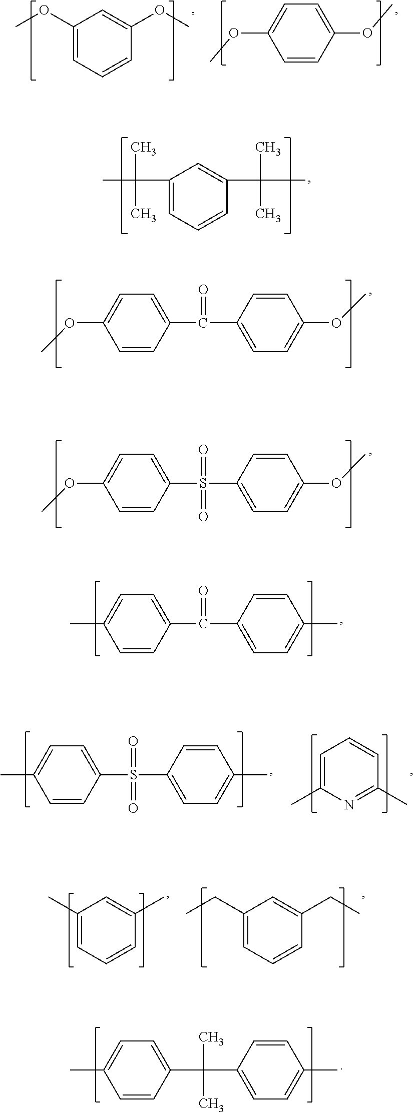

Further difunctional groups include, but are not limited to the following groups:

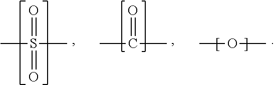

As used herein, the term “Glass transition temperature” or “Tg” means the temperature of reversible transition of an amorphous solid, e.g. polymer, between high elastic state and vitreous (glassy) state, when the polymer becomes brittle on cooling, or soft on heating. More specifically, it defines a pseudo second order phase transition, in which a supercooled melt yields, on cooling, a glassy structure and properties similar to those of crystalline materials, e.g. of an isotropic solid material.

The solubility of compounds in the present invention is determined as follows:
10 g of a sample are weighed into a 100 ml conical flask. 50 ml of a solvent are added to the flask and the mixture is stirred with magnetic bar for 1 h at 25° C. to assure the formation of a saturated solution. If the entire sample is dissolved, additional portion of the sample should be added and the mixture should be stirred further for 1 h. In the end, a portion of undissolved material should be clearly seen at the bottom of the flask. After that, the supematant is filtered through a folded filter. Some 15 g of the filtrate are weighed into a tared round-bottom flask, and the solvent is evaporated to dryness on a rotary evaporator at 90° C. under reduced pressure. Finally, the flask is dried in a vacuum drying cabinet at 120° C. for 2 h under reduced pressure, cooled down to room temperature in a desiccator and weighed.

The solubility value is then calculated as:

$$Solubility [ \% ] = \frac{[ Output weight ] \times 100}{[ Original sample weight ]}$$

### Bismaleimides according to the Invention

In a first aspect, the present invention refers to a bismaleimide according to formula (I)

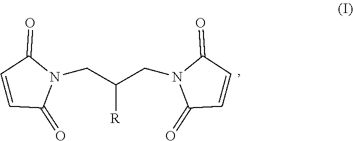

wherein
R is a substituted or unsubstituted C₃₋₇ cycloaliphatic ring, preferably a C₅₋₆ cycloaliphatic ring; or is a group of formula (II)

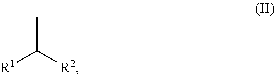

wherein R¹ and R² can be the same or different and are independently selected from C_{1-12,} preferably C₃₋₆ alkyl, or alkenyl groups. In preferred embodiment the C₃₋₇ cycloaliphatic ring is substituted with 1 to 5 C₁₋₄ alkyl groups.

In a preferred embodiment R of the bismaleimide of formula (I) is is a cyclohexyl group of formula (III)

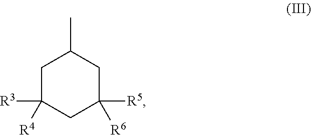

wherein R³ to R⁶ can be the same or different and are independently selected from H or C₁₋₃ alkyl groups.

In a preferred embodiment the bismaleimide is 2-(3,3,5-trimethylcyclohexyl)propane-1,3-bismaleimide having formula (IV)

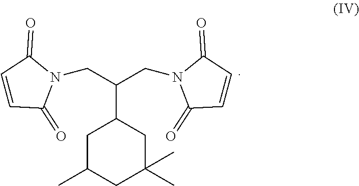 [SMILES: CC1CC(C(CN2C(=O)C=CC2=O)CN2C(=O)C=CC2=O)CC(C)(C)C1]

### Curable Compositions according to the Invention

In a second aspect, the present invention refers to a curable composition comprising
(i) at least one bismaleimide according to the present invention;
(ii) at least one polyimide of general formula (V)

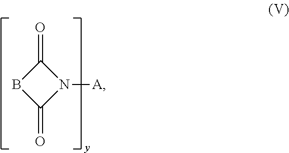

wherein
B is a difunctional group containing a carbon-carbon double bond, and
A is a y-functional group; and
y is an integer ≥2; and
(iii) at least one co-monomer or a combination of at least two co-monomers selected from:
(a) a compound of formula (VI)

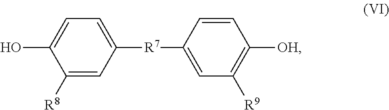

wherein
R⁷ is a difunctional group, and
R⁸ and R⁹ can be the same or different and are independently selected from alkenyl groups with 2 to 6 carbon atoms;
(b) a compound of formula (VII)

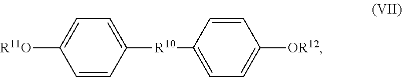

wherein
R¹⁰ is a difunctional group, and
R¹¹ and R¹² can be the same or different and are independently selected from alkenyl groups with 2 to 6 carbon atoms;
(c) a compound of formula (VIII)

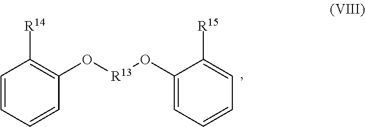

wherein
R¹³ is a difunctional group, and
R¹⁴ and R¹⁵ can be the same or different and are independently selected from alkenyl groups with 2 to 6 carbon atoms;
(d) a compound of formula (IX)

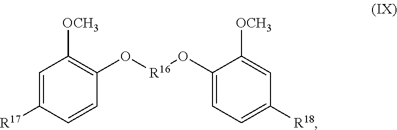

wherein
R¹⁶ is a difunctional group, and
R¹⁷ and R¹⁸ can be the same or different and are independently selected from alkenyl groups with 2 to 6 carbon atoms;
(e) a compound of formula (X)

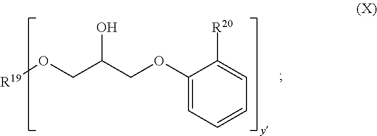

wherein
R¹⁹ is a y′-functional group, and
R²⁰ is an alkenyl group with 2 to 6 carbon atoms, and
y′ is an integer ≥2;
(f) a compound of formula (XI)

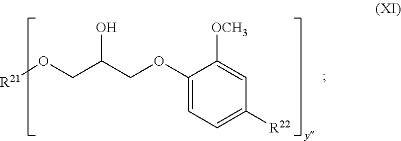

wherein
R²¹ is a y″-functional group, and
R²² is alkenyl group with 2 to 6 carbon atoms, and
y″ is an integer ≥2.

In one preferred embodiment the B in the polyimide of formula (V) is selected from the following difunctional groups:

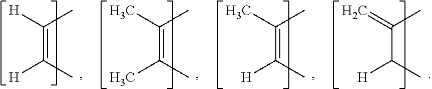

In one preferred embodiment A in the polyimide of formula (V) is selected from the following difunctional groups:
a) alkylene group with 2 to 12 carbon atoms;
b) cycloalkylene group with 5 to 6 carbon atoms;
c) heterocyclic group with 4 to 5 carbon atoms and at least one nitrogen, oxygen, or sulphur atom in the ring;
d) mono-or dicarbocyclic group;
e) bridged multicyclic group consisting of at least two groups selected from the following:
   monocarbocyclic aromatic groups, dicarbocyclic aromatic groups, cycloalkylene groups; wherein these groups are linked to each other by direct carbon-carbon bonds or by divalent groups; wherein preferably the divalent groups are selected from the following: oxy-group, thio-group, alkylene-group with 1 to 3 carbon atoms, sulfone-group, methanone-group, or one of the following groups

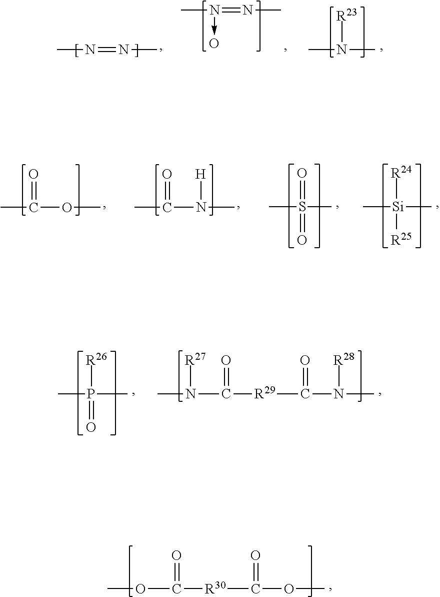

wherein R²³ to R²⁸ are independently selected from alkyl groups with 1 to 6 carbon atoms; and
R²⁹ and R³⁰ are independently selected from alkylene groups with 1 to 6 carbon atoms;
f) a group defined by formula (XII)

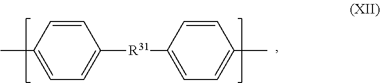

wherein R³¹ is one of the following groups

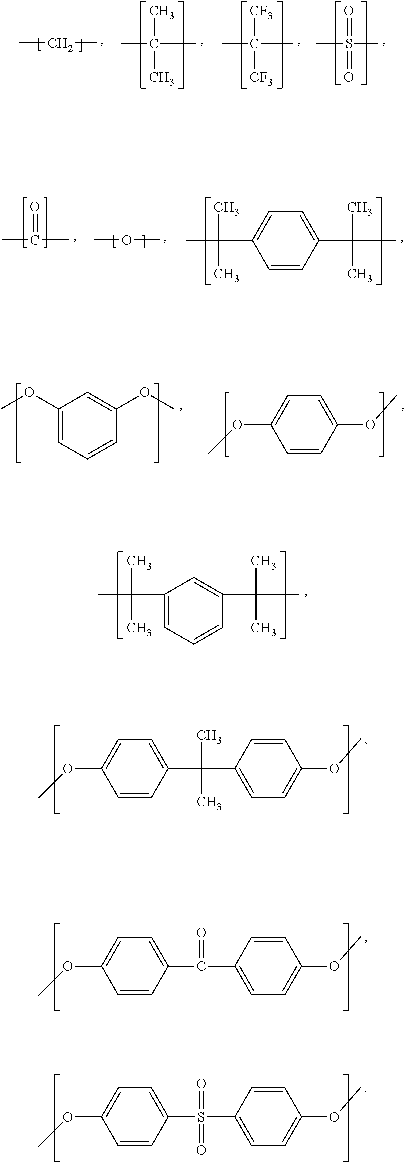

In one preferred embodiment the polyimide of formula (V) is a bisimide of formula (Va)

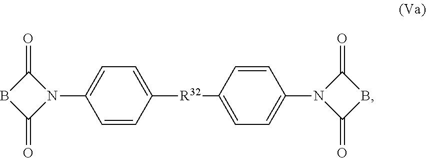

wherein R³² is one of the following groups

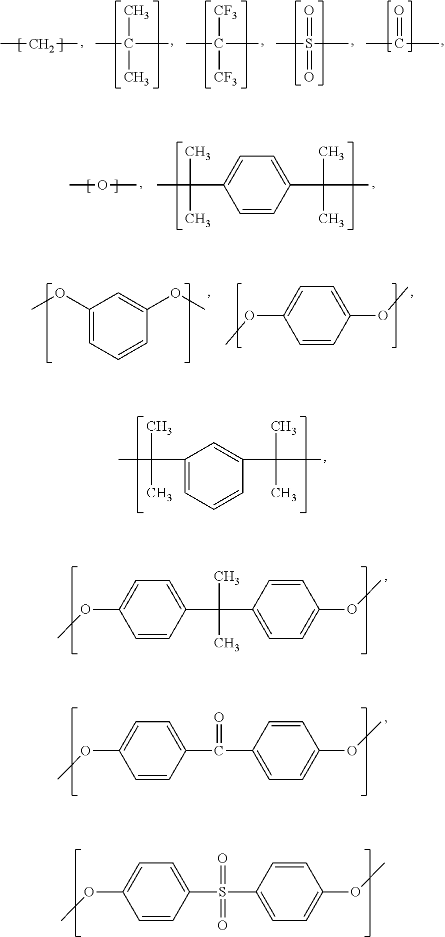

and B is defined as in formula (V).

In one preferred embodiment the at least one polyimide of formula (V) is a bismaleimide selected from: 4,4′-bismaleimidodiphenylmethane, bis(3-methyl-5-ethyl-4-maleimidophenyl)methane, bis(3,5-dimethyl-4-maleimidophenyl)methane, 4,4′-bismaleimidodiphenylether, 4,4′-bismaleimidodiphenylsulfone, 3,3′-bismaleimidodiphenylsulfone, bismaleimidodiphenylindane, 2,4-bismaleimidotoluene, 2,6-bismaleimidotoluene, 1,3-bismaleimidobenzene, 1,2-bismaleimidobenzene, 1,4-bismaleimidobenzene, 1,2-bismaleimidoethane, 1,6-bismaleimidohexane, 1,6-bismaleimido-(2,2,4-trimethyl)hexane, 1,6-bismaleimido-(2,4,4-trimethyl)hexane, 1,4-bis(maleimidomethyl)cyclohexane, 1,3-bis(maleimidomethyl)cyclohexane, 1,4-bismaleimidodicyclohexylmethane, 1,3-bis(maleimidomethyl)benzene, 1,4-bis(maleimidomethyl)benzene or a mixture thereof.

In one preferred embodiment the bismaleimide of formula (I) is 2-(3,3,5-trimethylcyclohexyl)propane-1,3-bismaleimide and the polyimide according to formula (V) is selected from: 4,4′-bismaleimidodiphenylmethane, bis(3-methyl-5-ethyl-4-maleimidophenyl)methane, bis(3,5-dimethyl-4-maleimidophenyl)methane, 4,4′-bismaleimidodiphenylether, 4,4′-bismaleimidodiphenylsulfone, 3,3′-bismaleimidodiphenylsulfone, bismaleimidodiphenylindane, 2,4-bismaleimidotoluene, 2,6-bismaleimidotoluene, 1,3-bismaleimidobenzene, 1,2-bismaleimidobenzene, 1,4-bismaleimidobenzene, 1,2-bismaleimidoethane, 1,6-bismaleimidohexane, 1,6-bismaleimido-(2,2,4-trimethyl)hexane, 1,6-bismaleimido-(2,4,4-trimethyl)hexane, 1,4-bis(maleimidomethyl)cyclohexane, 1,3-bis(maleimidomethyl)cyclohexane, 1,4-bismaleimidodicyclohexylmethane, 1,3-bis(maleimidomethyl)benzene, 1,4-bis(maleimidomethyl)benzene.

In one embodiment the curable compositions further comprise one or more cure inhibitors. Cure inhibitors retard the polymerisation reaction, thus modifying processability and storage stability of the compositions and intermediate products, such as prepregs, moulding compounds and resin solutions. Suitable cure inhibitors are hydroquinone, 1,4-naphthoquinone, ionole and phenothiazine, which are used at concentrations between 0.1 wt.-% and 2.0 wt.-%, based on the total weight of the composition. It is advantageous to dissolve the inhibitor in one of the components prior to the preparation of the mixture.

In one embodiment the curable compositions further comprise one or more cure accelerators. Cure accelerators accelerate the curing process. Typically cure accelerators are added in an amount of 0.01 wt.-% to 5 wt.-%, preferably in an amount of 0.1 wt.-% to 2 wt.-% based on the total weight of the curable composition. Suitable cure accelerators include ionic and free radical polymerization catalysts. Examples for free radical polymerization catalysts include (a) organic peroxides such as ditertiary butyl peroxide, diamylperoxide and t-butylperbenzoate and (b) azo compounds such as azobisisobutyronitrile. Examples of ionic catalysts are alkali metal compounds, tertiary amines such as triethylamine, dimethylbenzylamine, dimethylaniline, azabicyclooctane, heterocyclic amines such as quinoline, N-methylmorpholine, methylimidazole and phenylimidazole and phosphorous compounds such as triphenylphosphine and quaternary phosphonium halides. The cure accelerators can be admixed with the components of the curable composition either by a powder blending process or by a solvent blending process.

The curable composition can further comprise at least one co-monomer. In one embodiment, the at least one co-monomer is selected from:
2,2′-diallylbisphenol-A, bisphenol-A diallyl ether, bis(o-propenylphenoxy)benzophenone, m-aminobenzhydrazide, bisphenol-A dicyanate ester, diallyl phthalate, triallyl isocyanurate, triallyl cyanurate, styrene, divinylbenzene or a mixture thereof.

In one embodiment, the at least one co-monomer is selected from alkenylphenol, alkenylphenyl ether, alkenyl phenol ether, polyamine, aminophenol, aminoacid hydrazide, cyanate ester, diallyl phthalate, triallyl isocyanurate, triallyl cyanurate, styrene, divinylbenzene, wherein the co-monomer is preferably present in 1 wt.-% to 30 wt.-%, based on the total weight of the composition.

In one embodiment the molar ratio between the unsaturated imide groups and reactive alkenyl groups in the curable composition ranges from 1.0 to 0.1, e.g. from 1.0 to 0.2, from 1.0 to 0.3, from 1.0 to 0.4, from 1.0 to 0.5, from 1.0 to 0.6, from 1.0 to 0.7 or from 1.0 to 0.8. These ranges lead to desireable cure kinetics.

In one embodiment the curable composition further comprises at least one rection inhibitor. A reaction inhibitor improves the processability and storage stability before use. Suitable reaction inhibitors are hydroquinone, 1,4-naphthoquinone and phenothiazine which can be used at concentrations between 0.1 wt.-% and 2.0 wt.-%, based on the total weight of the composition. It is advantageous to dissolve the inhibitor in one of the components prior to the preparation of the composition.

In one embodiment the curable composition further comprises at least one reaction modifier selected from from alkenylphenol, alkenylphenyl ether, alkenyl phenol ether, polyamine, aminophenol, aminoacid hydrazide, cyanate ester, diallyl phthalate, triallyl isocyanurate, triallyl cyanurate, styrene, divinylbenzene or mixtures thereof. The reaction modifier can be present in 1 wt.-% to 30 wt.-%, based on the total weight of the composition. Of these, allyl-type components such as diallylbisphenol-A, bisphenol-A diallylether, diallylphthalate, triallylisocyanurate and triallylcyanurate are preferred. They can slow down polymerisation kinetics and therefore widen the processing window. Reaction modifier like styrene or divinylbenzene are very effective in concentrations between 10 wt.-% and 20 wt.-% but accelerate polymerisation kinetics, providing faster curing resins and lowering their polymerisation temperature. Therefore, reaction modifier are an additional tool to modify cure velocity of the curable compositions of the invention. In cases where such reaction modifier are used, it is advantageous to first blend the bismaleimide according to the invention with the reaction modifier in the required proportion and then, in a second step, dissolve the polyimide part of the mixture in this blend, if necessary at elevated temperature.

In one embodiment the curable compositions of the present invention can further include from 0.01 wt.-% to about 30 wt.-%, based on the total weight of the composition, of at least one thermoplastic polymer like a polyaryl ether, a polyaryl sulfone, a polyarylate, a polyamide, a polyaryl ketone, a polyimide different from formula (V), a polyimide-ether, a polyolefin, an ABS resin, a polydiene or diene copolymer or mixtures thereof. Thermoplastics such as polysulfons and phenoxy resins are particularly miscible with the curable compositions of the present invention, and may be used to adjust resin viscosity and control flow during cure. Thermoplastic polymers may also be added to improve the fracture toughness. Thermoplastic polymers can be added to the curable compositions as fine powders, or may be dissolved in either the bismaleimid according to formula (I) or in the reaction modifier.

In one embodiment the curable composition can comprise at least one catalyst. The catalyst can be present in an amount of 0.01 wt.-% to 5 wt.-%, preferably in an amount of 0.1 wt.-% to 2 wt.-%, based on the total weight of the curable composition. Suitable catalysts include ionic and free radical polymerization catalysts. Examples for free radical polymerization catalysts include (a) organic peroxides such as ditertiary butyl peroxide, diamylperoxide and t-butylperbenzoate and (b) azo compounds such as azobisisobutyronitrile. Examples of ionic catalysts are alkali metal compounds, tertiary amines such as triethylamine, dimethylbenzylamine, dimethylaniline, azabicyclooctane, heterocyclic amines such as quinoline, N-methylmorpholine, methylimidazole and phenylimidazole and phosphorous compounds such as triphenylphosphine and quatenary phosphonium halides. The catalysts can be admixed with the components of the curable composition or may be added during the processing either by a powder blending process or by a solvent blending process as described below.

### Processes for the Manufacture of Curable Compositions according to the Present Invention

In a third aspect, the present invention refers to a process for the manufacture of curable compositions according to the present invention, comprising the steps of blending the at least one polyimide and the at least one bismaleimide using a powder-, melt-, or solvent assisted blending process to obtain the curable composition. The curable composition can be a solid, low-melting, tacky or liquid curable composition.

### Solvent Blending Process

In one embodiment, the processes for the manufacture of curable compositions of the invention is a solvent blending process, comprising the step of:
dissolving the components of the curable composition, in a solvent or diluent, resulting in a stable solution that can be further processed to prepreg. Alternatively, the solvent or diluent can be stripped off afterwards to obtain a curable composition as a solvent-free mass (resin), which can further be used in various hot melt processing technologies.

In one embodiment step of dissolving is performed at temperatures above 30° C.

Suitable solvents and diluents are all customary inert organic solvents. They include but are not limited to ketones such as acetone, methylethylketone, cyclohexanone; glycol ethers such as methyl glycol, methyl glycol acetate, propylene glycol monomethyl ether (methyl proxitol), methyl proxitol acetate, diethylene glycol, and diethylene glycol monomethyl ether; toluene and xylene, preferably in combination with 1,3-dioxolane as a co-solvent.

In one embodiment, the solvent mixture comprises up to 50 wt.-%, preferably up to 40 wt.-% of ketones such as acetone, methylethylketone, cyclohexanone, or glycol ethers such as ethylene glycol ether, propylene glycol ether, butylene glycol ether, and their acetates based on the total weight of the solvent mixture.

In one embodiment, a solution of the curable composition of the invention comprises from 30 wt.-% to 70 wt.-%, preferably from 40 wt.-% to 60 wt.-% solvent, e.g. of 1,3-dioxolane, or solvent mixtures comprising 1,3-dioloxane, and the above-identified solvents. Such concentrations are typically used in industrial dip coating processes.

### Melt Blending Process

In one embodiment, the processes for the manufacture of curable compositions of the invention is a melt blending process. In one embodiment the melt blending is performed at a temperature from 70° C. to 250° C. In one preferred embodiment, the method is carried out at temperatures from 90° C. to 170° C., more preferred from 100° C. to 150° C. The curable compositions are obtained as a low melting masses (resins).

### Crosslinked Polymers of the Curable Compositions according to the Present Invention

In a further aspect the present invention refers to a crosslinked polymer obtainable from the curable composition according to the present invention by heating the curable composition to a temperature in the range of from 70° C. to 280° C.

It has been found that the curable compositions of the invention are useful for the preparation of crosslinked polymers.

In one embodiment the heating is carried out at temperatures from 90° C. to 260° C., preferably from 100° C. to 250° C.

### Composite Materials according to the Present Invention and Processes for their Manufacture

It has been found that curable compositions of the present invention are useful for the preparation of composite materials.

In a further aspect the present invention refers a process for the manufacture of a composite material comprising the steps of mixing a curable composition according to the present invention or a crosslinked polymer according to the present invention, with a fibrous or particulate reinforcement and curing the mixture.

In a final aspect, the present invention refers to a composite material obtainable by a process according the present invention.

In one embodiment the curing step can place under simultaneous shaping under pressure to obtain mouldings, laminates, adhesive bonds, and foams.

In one embodiment the the curing composition or the crosslinked polymer with a fibrous or particulate reinforcement can be processed by known methods of the powder moulding industry for producing mouldings, with curing taking place with simultaneous shaping under pressure. For these applications the curable compositions are admixed with fibrous or particulate reinforcement, in the following referred to as fillers as well, and optionally colorants and flame retardants. Ideal fillers for example are short glass fibers, short carbon fibers or aramid fibers, paticulate fillers such as quartz, silica, ceramics. metal powders and carbon powder. Depending on the technical application of the moulded article two or more different fillers may be used at the same time.

### Applications

In one embodiment the composite material is a fiber composite. For this application fillers, in particular fibers such as glass, carbon or aramid in the form of rovings, fabrics, short fiber mats, or felts are impregnated with the curable composition, employing a solution of the said curable composition to impregnate said reinforcements. After drying off the solvent a prepreg is left, which in the second phase may be cured at a temperature between 180° C. and 350° C., optionally under pressure.

### Melt Prepregs

In one embodiment the composite material is a fiber-reinforced composite obtained via a hot melt process. In order to obtain such fiber-reinforced composites the curable compositions are processed as hot melts to a resin film on a carrier foil, subsequently fillers, e.g., fibers, in the form of rovings or fabrics, are pressed into the molten resin film to form a prepreg. For this process curable compositions, which have a low viscosity at low temperature are advantageous in order to provide adequate impregnation of fiber rovings or fabric.

### Laminates

In one embodiment the composite material is a fiber laminate. Prepregs manufactured by either the solvent/solution-or the hot-melt process from glass-, carbon-or aramid fibers, in the form of fabriques or rovings, are stacked to provide a prepreg laminate, which subsequently is cured under pressure or in a vacuum bag at a temperature between 150° C. and 280° C. preferably between 170° C. and 260° C.

In one embodiment, the curable composition as defined above is mixed, e.g. applied onto or blended, with a fibrous or particulate reinforcement (filler) with the use of standard processing techniques, e.g. with the use of the hot melt or solution-based prepregging, resin transfer moulding (RTM), resin infusion moulding (RIM), filament winding (FW) or compounding techniques.

Curing may be carried out at temperatures ranging from 70° C. to 280° C., preferably at temperatures ranging from 80° C. to 270° C., more preferably at temperatures ranging from 90° C. to 260° C., most preferably at temperatures ranging from 100° C. to 250° C., preferably for a time sufficient to complete cure.

In one embodiment, the composite material is a fiber-reinforced composite. In one embodiment, the composite material is a particulate-filled composite.

In one embodiment, the present invention relates to a method for the preparation of a composite material comprising the steps of:
(a) preparing a curable composition as defined above,
(b) applying a curable composition as defined above onto a fibrous reinforcement or blending with a particulate filler,
(c) curing the curable composition as defined above at a temperature ranging from 70° C. to 280° C., preferably for a time sufficient to complete cure, and
(d) simultaneously applying pressure to obtain the composite material.

Process step c) may be carried out at temperatures ranging from 70° C. to 280° C., preferably at temperatures ranging from 80° C. to 270° C., more preferably at temperatures ranging from 90° C. to 260° C., most preferably at temperatures ranging from 100° C. to 250° C., preferably for a time sufficient to complete cure.

In the practice of process step c) the conversion of the curable compositions of the invention into the crosslinked (cured) polymer may be carried out, in the presence of a curing catalyst as defined above.

In the practice of process step d) shaping under pressure is performed to obtain the composites of the invention. Process steps c) and d) are preferably carried out simultaneously.

A preferred application of the curable compositions of the invention is resins for fiber-reinforced composites. In order to obtain such fiber composites the curable compositions of the invention are processed as hot melts to resin film on a carrier foil, which is subsequently used to prepare prepolymers by pressing fibers in the form of rovings or fabrics into the resin film. For this process curable compositions, which have a low viscosity at low temperature are advantageous in order to provide adequate impregnation of fiber rowings or fabric.

In one embodiment the composites of the present invention are fiber-reinforced laminates or copper clad laminates for applications in printed circuit boards.

### EXAMPLES

The following examples are intended to illustrate but not to limit the invention.

### EXAMPLES

### A. Preparation of 2-(3,3,5-trimethylcyclohexyl)propane-1,3-bismaleimide

### Example 1

The 2-(3,3,5-trimethylcyclohexyl)propane-1,3-bismaleimide was produced according to the following reaction scheme:

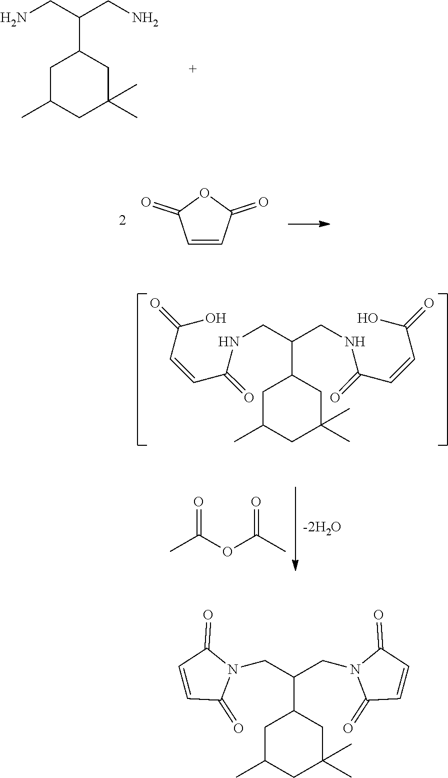

120 ml of N,N-dimethylacetamide were charged under nitrogen into a glass reactor equipped with mechanical stirrer, thermometer, and dropping funnel. 100 g of maleic anhydride were added and the mixture was stirred until dissolution was complete. Then, 99.2 g of 2-(3,3,5-trimethylcyclohexyl)propane-1,3-diamine were added dropwise so that the temperature did not exceed 60° C. After addition, the mixture was stirred for 1 hour at 50-55° C. Then, 128 g of acetic anhydride were added, followed by 200 g of triethylamine. The reaction mixture was heated up to 90° C., stirred for 1 hour, and cooled down to 60° C. The mixture was then stirred for 20 min at 60° C. and poured into 2 l of water under vigorous stirring. The precipitate was filtered off and washed by slurrying in distilled water. Finally, the product was filtered off and dried at 60° C. under reduced pressure. For analytical purposes, the product was purified by column chromatography using silica gel as a solid phase and methyl ethyl ketone as an eluent. M.p. 119° C. (DSC, 10° C./min).

¹H NMR (CDCl₃) δ 0.91/0.93* (s/s, 6H), 0.98 (d, 3H), 1.08 (t, 1H), 1.18 (dd, 1H), 1.30 (dd, 1H), 1.35 (d, 1H), 1.37-1.47 (m, 2H), 1.71 (m, 1H), 1.97 (m, 1H), 2.05 (m, 1H), 3.31/3.32* (d/d, 2H), 3.58* (m, 2H), 6.69/6.69* (s/s, 4H).

The resulting 2-(3,3,5-trimethylcyclohexyl)propane-1,3-bismaleimide is highly soluble in different organic solvents in comparison to other aliphatic bismaleimides:

TABLE 1
Solubilities of aliphatic bismaleimides in organic solvents,

| | Solubility at 25° C. in | | | | | | |
|---|---|---|---|---|---|---|---|
| | | | 1,3- | Ethyl | | Methyl | |
| BMI | Acetone | DMF^{a} | Dioxolane | acetate | MEK^{b} | proxitol^{c} | Toluene |
| 1,6-Bismaleimidohexane* | 2.6 | 6.1 | 8.7 | 2.1 | 2.6 | 3.3 | 1.5 |
| 1,3-Bismaleimidomethyl(cyclohexane) | 9.1 | 11.4 | 30.4 | 5.7 | 7.9 | 5.2 | 5.9 |
| 1,5-Bismaleimido(2-methylpentane) | 30.2 | 30.1 | 42.9 | 16.7 | 19.8 | 11.9 | 10.4 |
| 1,6-Bismaleimido-(2,2,4-/2,4,4,-trimethylhexane)* | 23.0 | 38.3 | 38.3 | 22.9 | 26.3 | 15.8 | 22.8 |
| mixture of isomers | | | | | | | |
| 2-(3,3,5-trimethylcyclohexyl)propane-1,3-bismaleimide | 43.7 | 42.6 | 54.6 | 34.6 | 47.9 | 38.0 | 40.4 |

*Commercial product;
^{a}N,N-dimethylformamide;
^{b}Methyl ethyl ketone;
^{c}1-Methoxy(2-propanol)

The solubility of the example and comparative examples was determined as follows:
10 g of a sample were weighed into a 100 ml conical flask. 50 ml of a solvent were added to the flask and the mixture was stirred with magnetic bar for 1 h at 25° C. to assure the formation of a saturated solution. If the entire sample was dissolved, additional portion of the sample should be added and the mixture should be stirred further for 1 h. In the end, a portion of undissolved material should be clearly seen at the bottom of the flask. After that, the supernatant was filtered through a folded filter. Some 15 g of the filtrate are weighed into a tared round-bottom flask, and the solvent was evaporated to dryness on a rotary evaporator at 90° C. under reduced pressure. Finally, the flask was dried in a vacuum drying cabinet at 120° C. for 2 h under reduced pressure, cooled down to room temperature in a desiccator and weighed.

The solubility value is then calculated as:

$$Solubility [ \% ] = \frac{[ Output weight ] \times 100}{[ Original sample weight ]}$$

### B. Preparation of Curable Mixtures according to the Invention Based on Bismaleimide of Formula (I), Polymaleimide of Formula (V), and a Co-Mononmer

The curable mixtures according to the invention can be obtained according to the following general processes:

### (a) Solvent-Assisted Process

At least one polymaleimide of formula (V) and at least one bismaleimide of formula (I) and, if required, at least one additional co-monomer component and an organic solvent, preferably toluene or methylene chloride, in a weight ratio solid-to-solvent of 1:1 are heated to 90-100° C. until a clear solution is obtained. Subsequently, the solvent is stripped off under reduced pressure, and the temperature is simultaneously increased to between 100-120° C. Finally, the mixture is degassed for 2-10 minutes under reduced pressure of 20 hPa [15 mm Hg] to obtain a curable mixture. The resin/solvent ratio may vary, depending on the solubility of components. Other solvents or diluents, as mentioned in the patent, may also be used.

### (b) Melt Process

At least one polymaleimide of formula (V), at least one bismaleimide of formula (I) and, if required, at least one additional co-monomer component are melt-blended in a temperature range of 100-120° C. until a homogeneous mixture is obtained. Subsequently, the melt thus obtained is further heated in the same temperature range for a time sufficient to obtain a stable melt. Finally, the melt is degassed under reduced pressure of 20 hPa [15 mm Hg] for 2-10 minutes to obtain the curable mixture.

### (c) Reactivity Measurements

### (c.1) Differential Scanning Calorimetry (DSC)

Differential scanning calorimetric (DSC) traces, obtained at a defined heating rate (10° C./min) in the temperature range from 20 to 380° C., are used to characterize the cure kinetics of curable compositions of the present invention. The cure exothermic maximum, T_{MAX}, represents the temperature of maximum heat release due to polymerization at the specified heating rate. The growth onset of the exothermic peak represents the temperature of polymerization onset, T_{ONSET}. The higher are T_{ONSET} and T_{MAX} the slower is the cure of a resin.

### (c.2) Hot-Plate Gel Time

Being a standard measure of resin reactivity, the gel time is measured by placing 1 g of the resin on an electrically heated metal block with a polished surface, which is capable of being maintained at temperatures between 130° C. and 230° C., and continuous stirring and probing the molten sample with a wooden rod, as described in the ISO 8987:2005-12 and ASTM D4217-07 (2017) norms.

### C. Curable Polymaleimide/Asymmetric Substituted Bis-Alkenyl Diphenyl Ether Mixtures

### Example 2

Curable mixture comprising 60 wt.-% bismaleimide of formula (IV), and 40 wt.-% 2,2′-bis(3-allyl-4-hydroxyphenyl)propane prepared by a solvent-assisted process (a) with the use of toluene as a solvent.
Gel time: 54 min.
Dynamic viscosity at 90° C.: 487 mPa·s; at 110° C.: 122 mPa·s.
DSC Polymerization onset (T_{ONSET}): 150° C.
DSC Polymerization maximum (T_{MAX}): 279° C.

### Example 3

Curable mixture comprising 35 wt.-%, bismaleimide of formula (IV), 35 wt.-% meta-xylylene bismaleimide, and 30 wt.-% 4,4′-bis(ortho-propenylphenoxy)benzophenone, prepared by a solvent-assisted process (a) with the use of toluene as a solvent.
Gel time: 48 min
Dynamic viscosity at 90° C.: 867 mPa·s; at 110° C.: 194 mPa·s.
DSC Polymerization onset (T_{ONSET}): 136° C.
DSC Polymerization maximum (T_{MAX}): 253° C.

### Example 4

Curable mixture comprising 30 wt.-% bismaleimide of formula (IV), 30 wt.-% 4,4′-bismaleimidodiphenylmethane, 26.7 wt.-% 4,4′-bis(ortho-propenyiphenoxy)benzophenone, and 13.3 wt.-% 2,2′-bis(3-allyl-4-hydroxyphenyl)propane, prepared by a solvent-assisted process (a) with the use of toluene as a solvent.
Gel time: 27 min
Dynamic viscosity at 90° C.: 2043 mPa·s; at 110° C.: 3302 mPa·s.
DSC Polymerization onset (T_{ONSET}): 135° C.
DSC Polymerization maximum (T_{MAX}): 260° C.

### Example 5

A mixture comprising 21 g of bismaleimide of formula (IV), 9 g of 2,2′-bis(3-allyl-4-hydroxyphenyl)propane, and 30 g of methyl ethyl ketone was stirred at 60° C. for 10 min, filtered, and cooled down to room temperature, providing a resin solution containing 50 wt.-% of solids. No crystallization was observed after six weeks at room temperature.
Gel time: 62 min
Dynamic viscosity: 17 mPa·s

1. A bismaleimide according to formula (I) 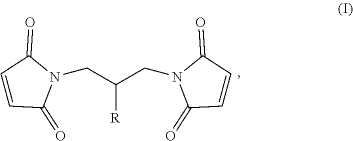
wherein R is a substituted or unsubstituted C₃₋₇ cycloaliphatic ring; or is a group of formula (II) 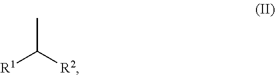
wherein R¹ and R² can be the same or different and are independently selected from the group consisting of C₂₋₁₂ and alkenyl groups.

2. The bismaleimide of claim 1, wherein R is a cyclohexyl group of formula (III) 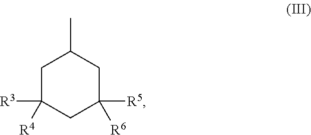
wherein R³ to R⁶ can be the same or different and are independently selected from the group consisting of H and C₁₋₃ alkyl groups.

3. The bismaleimide of claim 1, wherein the bismaleimide is 2-(3,3,5-trimethylcyclohexyl)propane-1,3-bismaleimide having formula (IV)

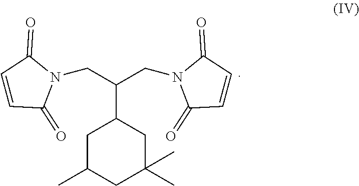 [SMILES: CC1CC(C(CN2C(=O)C=CC2=O)CN2C(=O)C=CC2=O)CC(C)(C)C1]

4. A curable composition, comprising:
(i) at least one bismaleimide according to claim 1;
(ii) at least one polyimide of general formula (V) 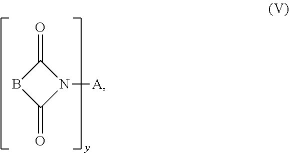
wherein
B is a difunctional group containing a carbon-carbon double bond,
A is a y-functional group; and
y is an integer ≥2; and
(iii) at least one co-monomer or a combination of at least two co-monomers selected from the group consisting of:
(a) a compound of formula (VI) 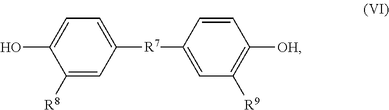
wherein
R⁷ is a difunctional group, and
R⁸ and R⁹ can be the same or different and are independently selected from alkenyl groups with 2 to 6 carbon atoms;
(b) a compound of formula (VII) 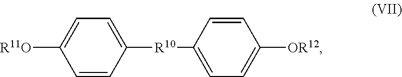
wherein
R¹⁰ is a difunctional group, and
R¹¹ and R¹² can be the same or different and are independently selected from alkenyl groups with 2 to 6 carbon atoms;
(c) a compound of formula (VIII) 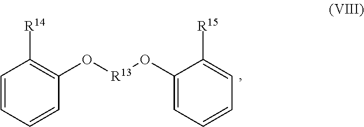
wherein
R¹³ is a difunctional group, and
R¹⁴ and R¹⁵ can be the same or different and are independently selected from alkenyl groups with 2 to 6 carbon atoms;
(d) a compound of formula (IX) 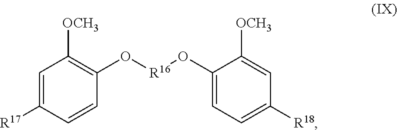
wherein
R¹⁶ is a difunctional group, and
R¹⁷ and R¹⁸ can be the same or different and are independently selected from alkenyl groups with 2 to 6 carbon atoms;
(e) a compound of formula (X) 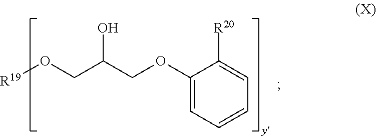
wherein
R¹⁹ is a y′-functional group,
R²⁰ is an alkenyl group with 2 to 6 carbon atoms, and
y′ is an integer ≥2; and
(f) a compound of formula (XI) 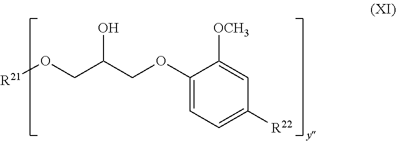
wherein
R²¹ is a y″-functional group,
R²² is alkenyl group with 2 to 6 carbon atoms, and
y″ is an integer ≥2.

5. Curable The curable composition according to claim 4,
wherein B in the polyimide of formula (V) is a difunctional group selected from the group consisting of:

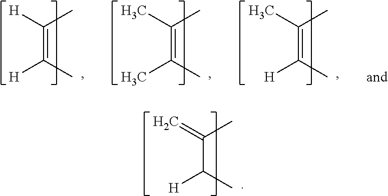

6. The curable composition according to claim 4, wherein the A in the polyimide of formula (V), is a difunctional group selected from the group consisting of:
a) an alkylene group with 2 to 12 carbon atoms;
b) a cycloalkylene group with 5 to 6 carbon atoms;
c) a heterocyclic group with 4 to 5 carbon atoms and at least one nitrogen, oxygen, or sulphur atom in the ring;
d) a mono-or dicarbocyclic group;
e) a bridged multicyclic group consisting of at least two groups selected from the following: group consisting of monocarbocyclic aromatic groups, dicarbocyclic aromatic groups, and cycloalkylene groups; wherein these groups are linked to each other by direct carbon-carbon bonds or by divalent groups;
f) a group defined by formula (XII) 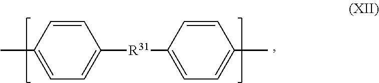
wherein R³¹ is one of the following groups

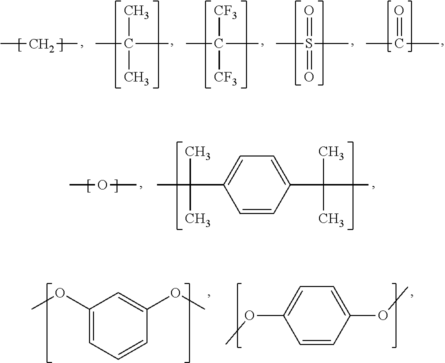

7. The curable composition according to claim 4, wherein the polyimide of formula (V) is a bisimide of formula (Va) 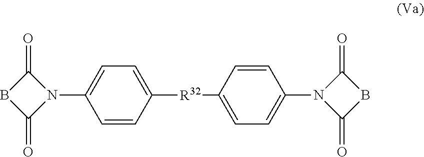
wherein R³² is one of the following groups

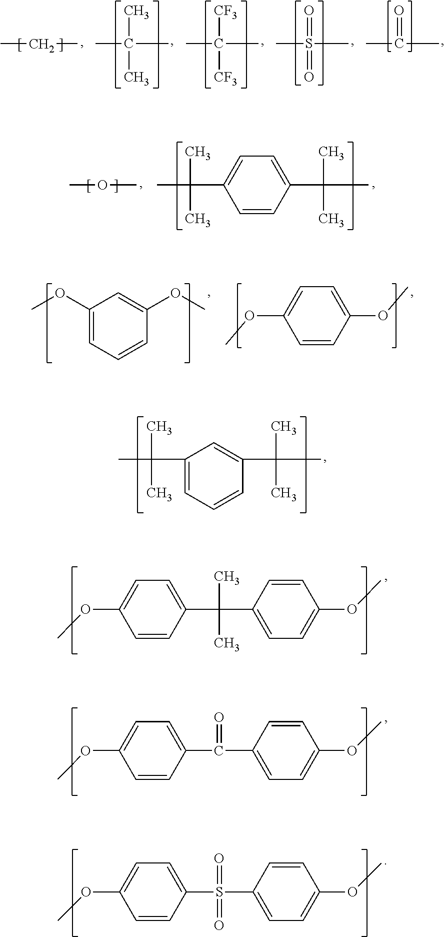

8. The curable composition according to claim 4, wherein the at least one polyimide of formula (III) is a bismaleimide selected from the group consisting of 4,4′-bismaleimidodiphenylmethane, bis(3-methyl-5-ethyl-4-maleimidophenyl)methane, bis(3,5-dimethyl-4-maleimidophenyl)methane, 4,4′-bismaleimidodiphenylether, 4,4′-bismaleimidodiphenylsulfone, 3,3′-bismaleimidodiphenylsulfone, bismaleimidodiphenylindane, 2,4-bismaleimidotoluene, 2,6-bismaleimidotoluene, 1,3-bismaleimidobenzene, 1,2-bismaleimidobenzene, 1,4-bismaleimidobenzene, 1,2-bismaleimidoethane, 1,6-bismaleimidohexane, 1,6-bismaleimido-(2,2,4-trimethyl)hexane, 1,6-bismaleimido-(2,4,4-trimethyl)hexane, 1,4-bis(maleimidomethyl)cyclohexane, 1,3-bis(maleimidomethyl)cyclohexane, 1,4-bismaleimidodicyclohexylmethane, 1,3-bis(maleimidomethyl)benzene, 1,4-bis(maleimidomethyl)benzene and a mixture thereof.

9. A process for the manufacture of curable compositions according to claim 4, comprising:
blending the at least one polyimide and the at least one bismaleimide using a powder-, melt-, or solvent assisted blending process to obtain the curable composition.

10. A crosslinked polymer obtainable from the curable composition according to claim 4 by heating the curable composition to a temperature in a range of from 70° C. to 280° C.

11. A process for the manufacture of a composite material, comprising:
mixing the curable composition according to claim 4, with a fibrous or particulate reinforcement. and
curing the mixture.

12. A composite material obtainable by the process according to claim 11.

13. The curable composition according to claim 6, wherein the divalent groups are selected from the group consisting of oxy-groups, thio-groups, alkylene-groups with 1 to 3 carbon atoms, sulfone-groups, methanone-groups, and the following groups: 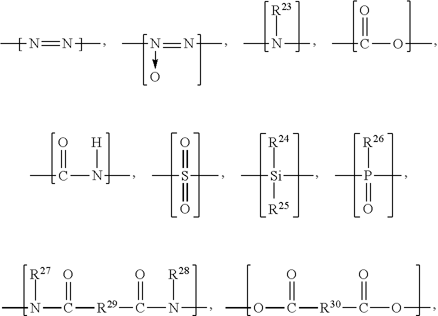
wherein R²³ to R²⁸ are independently selected from alkyl groups with 1 to 6 carbon atoms; and
R²⁹ and R³⁰ are independently selected from alkylene groups with 1 to 6 carbon atoms.

14. A process for the manufacture of a composite material, comprising:
mixing the crosslinked polymer according to claim 10 with a fibrous or particulate reinforcement, and
curing the mixture.
